(19) United States
(12) Patent Application Publication
(10) Pub. No.: US 2015/0185197 A1
(43) Pub. Date: Jul. 2, 2015

(54) METHOD FOR MEASURING DILUTION AND VISCOSITY OF LUBRICATING OIL, CONTROL METHOD AND CONTROL MODULE, AND REFRIGERATION AIR CONDITIONING SYSTEM

(71) Applicant: Danfoss (Tianjin) Ltd., Tianjin (CN)

(72) Inventors: Fan Liang, Tianjin (CN); Sun Zili, Tianjin (CN); Ding Siyuan, Tianjin (CN); Huang Zhigang, Tianjin (CN); Li Jingyuan, Tianjin (CN); Sun Yingke, Tianjin (CN); Wang Dong, Tianjin (CN); Zhang Leping, Tianjin (CN)

(21) Appl. No.: 14/581,126

(22) Filed: Dec. 23, 2014

(30) Foreign Application Priority Data
Dec. 31, 2013 (CN) 201310756021.1

(51) Int. Cl. G01N 33/28; G01N 7/00; F25B 31/00; G01N 25/00; F25B 1/00; F25B 49/02

(52) U.S. Cl. CPC G01N 33/2888; F25B 1/005; F25B 49/022; F25B 31/002; G01N 25/00; G01N 7/00

(57) ABSTRACT

Embodiments of the present invention provide a method for measuring dilution and/or viscosity of lubricating oil in a compressor. The method includes: detecting a pressure difference between an inlet and an outlet of an oil pump in the compressor by pressure difference detection apparatus, and determining the dilution of the lubricating oil and/or the viscosity of the lubricating oil according to the pressure difference detected; a lower end of the oil pump being located in the lubricating oil in the oil sump, a high-pressure side of the pressure difference detection apparatus being connected to the outlet of the oil pump and a low-pressure side of the pressure difference detection apparatus being connected to bottom of the oil sump. Embodiments of the present invention further provides a method for detecting dilution of lubricating oil, a control method and control module for controlling a compressor, and a refrigeration air conditioning system.

### CROSS REFERENCE TO RELATED APPLICATIONS

Applicant hereby claims foreign priority benefits under U.S.C. §119 from Chinese Patent Application Serial No. CN201310756021.1 filed on Dec. 31, 2013, the contents of which are incorporated by reference herein.

### FIELD OF THE INVENTION

The present invention relates to the technical field of refrigeration air conditioning, and more particularly, to a method for measuring dilution and/or viscosity of lubricating oil in a compressor, a method for detecting dilution of lubricating oil in a compressor, a control method for controlling a compressor, and a control module for controlling a compressor, and a refrigeration air conditioning system.

### BACKGROUND OF THE INVENTION

During operation of a refrigeration air conditioning system, an excessive amount of refrigerant may return to a compressor due to some aspects such as significant changes of system load, improper control, a start process or a system defrosting action. As a result, lubricating oil in the compressor may be diluted, which reduces viscosity of the lubricating oil, leads to undesirable lubrication of operating parts in the compressor and thereby results in a malfunction of the compressor.

Currently, common methods for detecting dilution of lubricating oil include viscosity detection, density detection, and light absorptivity or a refractive index detection, in order to determine the amount of refrigerant in the lubricating oil and obtain a dilution level of the lubricating oil. Currently, common methods for detecting viscosity of lubricating oil include, for example, ultrasonic detection and tuning fork vibration viscometer measurement. These detection methods not only have very high costs and complex installation, but also require a huge data processing system. These detection methods are mostly limited in laboratory research, and are not applicable in engineering applications.

In addition, the viscosity of the lubricating oil in the compressor is affected by elements such as an ambient temperature, pressure and refrigerant solubility. When the compressor stops operation, oil pressure in an oil sump (that is, the pressure of the lubricating oil in the oil sump) is constant, and the viscosity of the lubricating oil decreases as the temperature drops down. Especially, when the ambient temperature is very low and the compressor starts operation under such ambient temperature, the refrigerant, instead of the lubricating oil, may be supplied to a bearing, which is dangerous to lubrication of the bearing.

In some situations, for a low-pressure chamber compressor, when the temperature of the compressor is lower than a temperature of a condensing unit or a condenser, the refrigerant flows to the compressor. This will bring similar danger to the lubrication of the bearing. Although the compressor runs at a low-pressure side, a large amount of liquid may flow into an oil sump under a situation of low evaporating temperature, which causes a rapid drop of the temperature in the oil sump and also a rapid decrease of oil viscosity. Once such a situation occurs, the compressor need be stopped or the oil sump may need be heated by a crankcase heater.

The viscosity of the lubricating oil is affected by the temperature, pressure and solubility of the lubricating oil mixed with the refrigerant. When the temperature of the oil sump is low, the oil viscosity decreases and sometimes the refrigerant may nearly occupy the entire bottom layer of the oil sump. This is also very dangerous to the lubrication of the bearing, thereby bringing a reliability problem to the compressor.

However, the existing heating solution by using a crankcase heater has a reliability problem.

When the ambient temperature is low and when the temperature of the compressor is lower than the temperature of the condensing unit or a large amount of liquid flows into the oil sump, a crankcase heater is usually used for heating the oil sump to increase the oil viscosity. The crankcase heater is controlled by a control apparatus or a driving apparatus of the compressor. Whether to turn on or off the crankcase heater is determined according to the ambient temperature. When the ambient temperature is low, the crankcase heater is turned on to heat the oil sump. However, the real status (for example, viscosity) of the lubricating oil cannot be detected, and it is thus possible that the oil sump is still being heated even if the viscosity of the lubricating oil is desirable enough. As a result, power consumption of the compressor is increased and the performance of the compressor is decreased.

### SUMMARY OF THE INVENTION

Embodiments of the present invention are to solve at least one aspect of the foregoing problems.

An embodiment of the present invention provides a method for determining dilution and/or viscosity of lubricating oil more simply and quickly.

Another embodiment of the present invention provides a control method, which controls, according to a practical status of oil in an oil sump of a compressor, whether to heat the oil sump by using a crankcase heater.

According to an aspect of the present invention, a method for measuring dilution and/or viscosity of lubricating oil in a compressor includes:

detecting a pressure difference between an inlet and an outlet of an oil pump in the compressor by pressure difference detection apparatus, and determining the dilution of the lubricating oil and/or the viscosity of the lubricating oil according to the pressure difference detected; a lower end of the oil pump being located in the lubricating oil in the oil sump, a high-pressure side of the pressure difference detection apparatus being connected to the outlet of the oil pump and a low-pressure side of the pressure difference detection apparatus being connected to bottom of the oil sump.

In an implementation manner, the oil pump is a positive displacement oil pump.

In an implementation manner, the pressure difference detection apparatus is a pressure difference transmitter, the high-pressure side of the pressure difference transmitter is connected to the outlet of the oil pump through a high-pressure pipe, and a low-pressure side of the pressure differential transmitter is connected to the bottom of the oil sump through a low-pressure pipe.

In an implementation manner, the detecting the pressure difference between the inlet and the outlet of the oil pump in the compressor is performed under multiple different operating conditions, and accordingly the determining the dilution and/or the viscosity of the lubricating oil according to the pressure difference is performed respectively under the multiple different operating conditions; the method further includes:

obtaining a relationship curve between the pressure difference and the dilution of the lubricating oil by way of fitting, and/or a relationship curve between the pressure difference and the viscosity of the lubricating oil by way of fitting.

In an implementation manner, the method further includes:

after detecting the pressure difference between the inlet and the outlet of the oil pump, obtaining the dilution and/or the viscosity of the lubricating oil corresponding to the pressure difference detected according to the relationship curve(s).

In an implementation manner, the compressor is a low-pressure chamber compressor or a high-pressure chamber compressor used in a refrigeration air conditioning system.

According to another aspect of the present invention, method for detecting dilution of lubricating oil in a compressor includes:

measuring an oil sump temperature of a compressor, an evaporating/condensing temperature of the compressor, and dilution of the lubricating oil under each of multiple different operating conditions;

calculating oil sump superheat according to an equation that the oil sump superheat=the oil sump temperature−the evaporating/condensing temperature;

obtaining, by way of fitting, a relationship curve between the oil sump superheat and the dilution of the lubricating oil; and

obtaining, according to the relationship curve between the oil sump superheat and the dilution of the lubricating oil, dilution of the lubricating oil corresponding to oil sump superheat obtained under a certain operating condition.

In an implementation manner, the condensing temperature of the compressor is a saturation temperature corresponding to discharge pressure of the compressor, and the evaporating temperature of the compressor is a saturation temperature corresponding to suction pressure of the compressor.

In an implementation manner, the saturation temperature corresponding to the suction pressure of the compressor or corresponding to the discharge pressure of the compressor is calculated by a thermophysical property equation or diagram of refrigerant in the compressor.

In an implementation manner, the evaporating temperature of the compressor is a midpoint temperature of an evaporator coil.

In an implementation manner, the condensing temperature of the compressor is midpoint temperature of a condenser coil.

In an implementation manner, the relationship curve between the oil sump superheat and the dilution of the lubricating oil is: y=(0.0003x2−0.0233x+0.5979)−a, y represents the dilution, x represents the oil sump superheat, and a represents a modification coefficient.

In an implementation manner, the relationship curve between the oil sump superheat and the dilution of the lubricating oil is written in control software of a control panel of the compressor, and the dilution of the lubricating oil is obtained on the control panel according to the oil sump superheat obtained through calculation;

wherein the method may further include: displaying by the control panel the dilution of the lubricating oil obtained.

In an implementation manner, the relationship curve between the oil sump superheat and the dilution of the lubricating oil is written in control software of a display device, the display device being externally connected to the compressor and serving as an accessory of the compressor; the method may further include: displaying by the display device the dilution of the lubricating oil according to the oil sump superheat obtained through calculation.

According to yet another aspect of the present invention, a control method operable for controlling a compressor includes:

obtaining viscosity of lubricating oil in an oil sump of the compressor; and

controlling the compressor to be turned on or off or controlling a crankcase heater of the compressor to be turned on or off according to the viscosity.

In an implementation manner, the obtaining the viscosity of the lubricating oil in the oil sump of the compressor includes: obtaining the viscosity of the lubricating oil in the oil sump of the compressor according to the above method.

In an implementation manner, the obtaining the viscosity of the lubricating oil in the oil sump of the compressor comprises: detecting an oil sump temperature in the compressor, detecting oil sump pressure in the compressor, and obtaining the viscosity of the lubricating oil in the oil sump according to the oil sump temperature and the oil sump pressure.

According to yet another aspect of the present invention, a control module operable for controlling a compressor includes:

a viscosity obtaining unit, operable for obtaining viscosity of lubricating oil in an oil sump of the compressor; and

a control unit, operable for controlling according to the viscosity of the lubricating oil the compressor to be turned on or off or controlling according to the viscosity of the lubricating oil a crankcase heater of the compressor to be turned on or off.

In an implementation manner, the viscosity obtaining unit may include:

a temperature signal receiving unit, operable for receiving an oil sump temperature of the compressor;

a pressure signal receiving unit, operable for receiving oil sump pressure of the compressor; and

a calculation unit, operable for obtaining the viscosity of the lubricating oil in the oil sump according to the oil sump temperature and the oil sump pressure.

According to yet another aspect of the present invention, a refrigeration air conditioning system is provided. The refrigeration air conditioning system includes a compressor, a condenser, throttling apparatus, and an evaporator which are sequentially connected through a pipeline, and a refrigerant goes through evaporation, compression, condensing, and throttling in the refrigeration air conditioning system in a refrigeration circulation; the refrigeration air conditioning system may further includes a crankcase heater, and the crankcase heater is controlled to be turned on or off according to viscosity of lubricating oil in an oil sump of the compressor.

In an implementation manner, the refrigeration air conditioning system may further include a temperature sensor operable for detecting oil sump temperature, and the temperature sensor is installed inside the oil sump or around an external sidewall or at a lower end of the outside of the oil sump.

In an implementation manner, the oil sump temperature is indirectly or approximately obtained from a discharge/suction temperature of the compressor.

In an implementation manner, oil sump pressure is directly obtained from suction pressure of the compressor.

In an implementation manner, the viscosity of the lubricating oil in the oil sump is obtained according to the oil sump temperature and the oil sump pressure; the crankcase heater is operable to start to heat the oil sump to increase the viscosity of the lubricating oil when the viscosity of the lubricating oil in the oil sump is lower than a preset value.

In an implementation manner, the throttling apparatus may be an expansion valve or a capillary tube.

In an implementation manner, the crankcase heater may be controlled according to the above-mentioned control method or by the above-mentioned control module.

### BRIEF DESCRIPTION OF THE DRAWINGS

Other aspects and advantages of the present invention will be described in detail in the following description of embodiments with reference to the accompanying drawings:

FIG. 1 is a schematic diagram of principle of pressure difference detection apparatus in a compressor in accordance with a first embodiment of the present invention;

FIG. 2 shows a measurement curve and a fitting curve of a pressure difference between an inlet and an outlet of an oil pump in the compressor and viscosity of lubricating oil according to the principle shown in FIG. 1 in accordance with an embodiment of the present invention;

FIG. 3 shows a measurement curve and a fitting curve of the pressure difference between the inlet and the outlet of the oil pump in the compressor and dilution of the lubricating oil according to the principle shown in FIG. 1 in accordance with an embodiment of the present invention;

FIG. 4 shows a measurement curve and a fitting curve of a temperature of the oil sump in the compressor and dilution of lubricating oil in the oil sump according to a second embodiment of the present invention;

FIG. 5a is a schematic diagram of a refrigeration air conditioning system in accordance with a third embodiment of the present invention;

FIG. 5b is a schematic diagram of a compressor having a crankcase heater shown in FIG. 5a in accordance with an embodiment of the present invention;

FIG. 6 is a curve diagram of pressure-temperature-solubility-in-refrigerant-viscosity of lubricating oil in the compressor in the refrigeration air conditioning system shown in FIG. 5a in accordance with an embodiment of the present invention;

FIG. 7 is a curve diagram of viscosity-temperature-solubility-in-refrigerant of the lubricating oil in the compressor in the refrigeration air conditioning system shown in FIG. 5a in accordance with an embodiment of the present invention; and

FIG. 8 is a curve diagram of pressure-temperature-solubility-in-refrigerant of the lubricating oil in the compressor in the refrigeration air conditioning system shown in FIG. 5a in accordance with an embodiment of the present invention.

### DETAILED DESCRIPTION OF THE PRESENT INVENTION

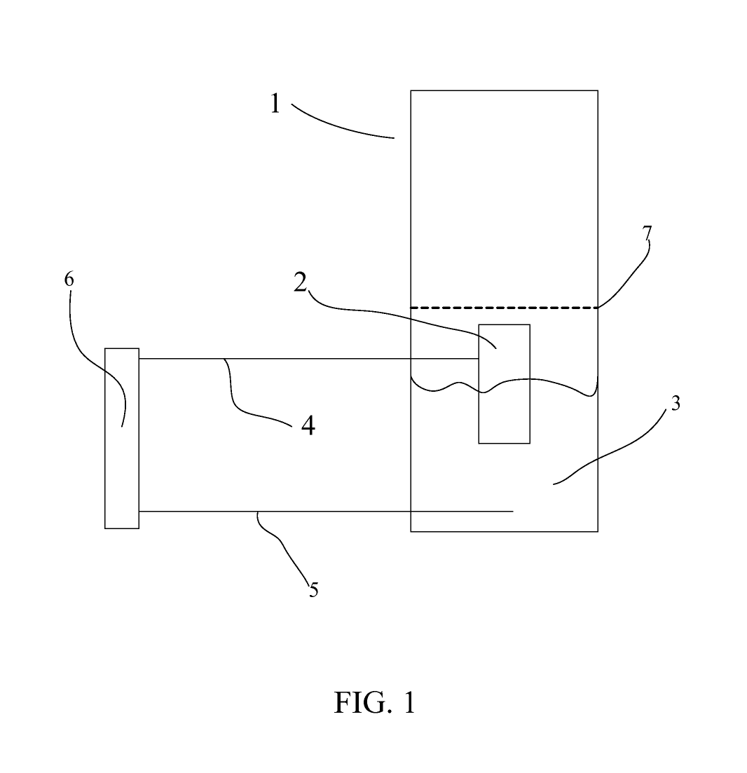

The technical solutions of the present invention are further described in detail below with reference to embodiments and FIG. 1 to FIG. 8. In the description, same or similar reference signs indicate same or similar members. The description of the implementation manners of the present invention below with reference to the accompanying drawings is intended to explain the general inventive concept of the present invention, and should not be construed as a limitation to the present invention.

### First Embodiment

As shown in FIG. 1, in a low-pressure chamber compressor 1, an oil pump 2 (for example, a positive displacement oil pump 2) is used and a lower end of the oil pump 2 is located in lubricating oil 3 in an oil sump 7 of the compressor. A wavy line shown in FIG. 1 is used to schematically represent an oil level in the lubricating oil 3. It may be understood that the size of the oil sump 7 of the compressor may be specifically determined according to practical requirements although the oil sump 7 of the compressor is specifically shown in FIG. 1, as long as the lubricating oil in the compressor can be accommodated.

An outlet of the oil pump 2 is, for example, connected to a pressure difference detection apparatus 6 (for example, a high-pressure side of a pressure difference detector) through a high-pressure pipe 4, and a low-pressure side of the pressure difference detection apparatus 6 is, for example, connected to the bottom of the oil sump 7 of the compressor through a low-pressure pipe 5. If the lubricating oil 3 is diluted, viscosity of the lubricating oil is reduced. Thereby, an oil discharge resistance of the positive displacement oil pump 2 is decreased. Accordingly, the pressure at a high-pressure side of the oil pump 2 is decreased, and the pressure at a low pressure side does not change much because the low-pressure side is located in the oil sump 7. As a result, a pressure difference between an inlet and outlet of the oil pump 2 and detected by the pressure difference detection apparatus 6 decreases, and thus changes of dilution and viscosity of the lubricating oil in the oil sump 7 may be determined according to the change of the pressure difference.

In practical applications, if two much refrigerant is dissolved in the lubricating oil, the lubricating oil is diluted and the viscosity of the lubricating oil is reduced. When the positive displacement oil pump 2 is installed in the compressor 1, the change of the pressure difference between the inlet and the outlet of the oil pump 2 may indirectly reflect changes of dilution and viscosity of the lubricating oil.

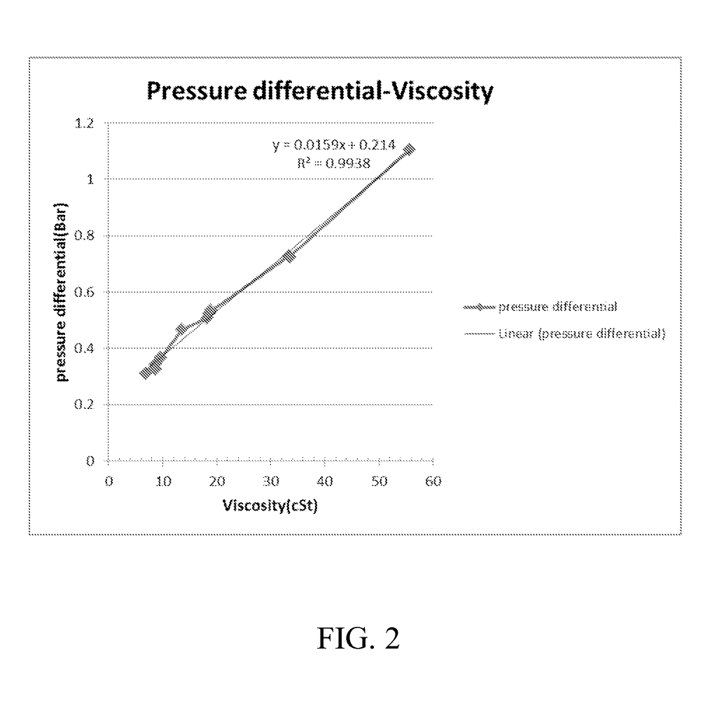

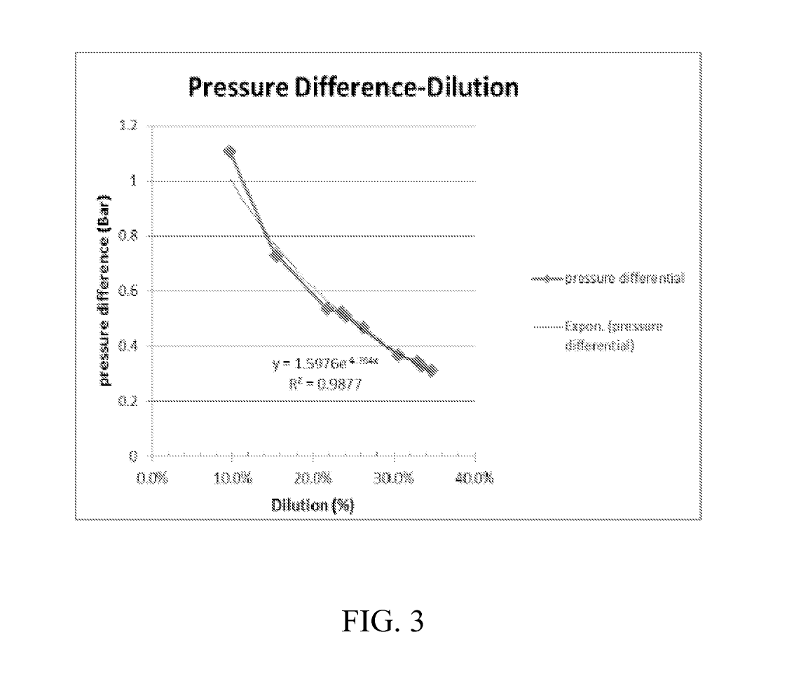

The inventor of the present invention finds it is a good idea to use the pressure difference to detect the dilution and/or viscosity of the lubricating oil, and has performed experiments in different operating conditions according to the principle shown in FIG. 1 to measure a pressure difference between the inlet and the outlet of the oil pump 2 as well as the dilution and viscosity of the lubricating oil in the compressor 1, and accordingly has obtained a curve of corresponding pressure difference and viscosity of lubricating oil (as shown in FIG. 2) and a curve of pressure difference and dilution of lubricating oil (as shown in FIG. 3). The inventor of the present invention finds that in the example shown in FIG. 2, the pressure difference and the viscosity of the lubricating oil have a linear relationship y=0.0159x+0.214, where y represents the pressure difference, x represents viscosity, and correlation coefficient R²=0.9938. When R² approximates 1, it indicates that the equation obtained by way of fitting is more accurate and reliable. Therefore, the pressure difference can be used to obtain the viscosity of the lubricating oil. For another example, the inventor of the present invention further finds that in the example shown in FIG. 3, the pressure difference and the dilution of the lubricating oil have an exponential relationship y=1.5976e^{-4.764x} where x represents the dilution of the lubricating oil, y represents the pressure difference, and correlation coefficient R²=0.9877. Therefore, it is possible to use the pressure difference to obtain the viscosity. However, the fitting relationships shown in FIG. 2 and FIG. 3 are only exemplary, other suitable relationships may be used according to requirements, and the present invention is not limited to the fitting relationships shown in FIG. 2 and FIG. 3. The inventor of the present invention finds, through the experiments, that the pressure difference and the viscosity always meet a very desirable corresponding relationship, and proposes a method for obtaining the viscosity according to the pressure difference.

After obtaining the fitting relationships, a pressure difference between the inlet and the outlet of the oil pump 2 is measured, and the viscosity and the dilution of the lubricating oil can be obtained respectively by looking up in respective tables generated according to the relationship between the pressure difference and the viscosity of the lubricating oil in FIG. 2 and the relationship between the pressure difference and the dilution of lubricating oil in FIG. 3.

The compressor in an embodiment of the present invention may not only be the low-pressure chamber compressor used in a refrigeration air conditioning system, but may also be a high-pressure chamber compressor used in the refrigeration air conditioning system.

As mentioned above, in the first embodiment of the present invention, the oil pump is configured inside the compressor and then the dilution and viscosity of lubricating oil can be determined simply and rapidly. The method is simple, low in cost, and applicable for practical applications in engineering.

### Second Embodiment

Solubility of the refrigerant in the lubricating oil (referred to as dilution of lubricating oil) depends on two factors: the temperature of the lubricating oil and the pressure of the lubricating oil. Oil sump superheat also depends on two factors: the temperature of the lubricating oil, and a saturation temperature or evaporating/condensing temperature corresponding to a suction/discharge pressure. Therefore, the inventor of the present invention finds that a certain correlation exists between the solubility of in the refrigerant the lubricating oil (i.e., the dilution of the lubricating oil) and oil sump superheat, and the correlation is further demonstrated by experiments.

The second embodiment of the present invention provides a method for detecting dilution of lubricating oil in a compressor used in a refrigeration air conditioning system. The method is described below. Under multiple different operating conditions, an oil sump temperature in a compressor is measured, an evaporating/condensing temperature of the compressor and dilution of lubricating oil are also measured. The oil sump superheat is calculated according to an equation that the oil sump superheat=the oil sump temperature−the evaporating/condensing temperature. A relationship curve between the oil sump superheat and the dilution of the lubricating oil is obtained through fitting. For the oil sump superheat obtained by calculation under an operating condition, corresponding dilution of the lubricating oil can be obtained by looking up in a table generated according to the relationship curve between the oil sump superheat and the dilution of the lubricating oil or according to the relationship curve between the oil sump superheat and the dilution of the lubricating oil.

The oil sump superheat is obtained by subtracting the evaporating/condensing temperature (or the saturation temperature corresponding to the suction/discharge pressure) from the oil sump temperature. It can be known that the condensing temperature of the compressor is the saturation temperature corresponding to the discharge pressure of the compressor, and the evaporating temperature of the compressor is the saturation temperature corresponding to the suction pressure of the compressor. For example, the saturation temperature corresponding to the suction pressure of the compressor or corresponding to the discharge pressure of the compressor may be calculated by using a thermophysical property equation or diagram of refrigerant in the compressor.

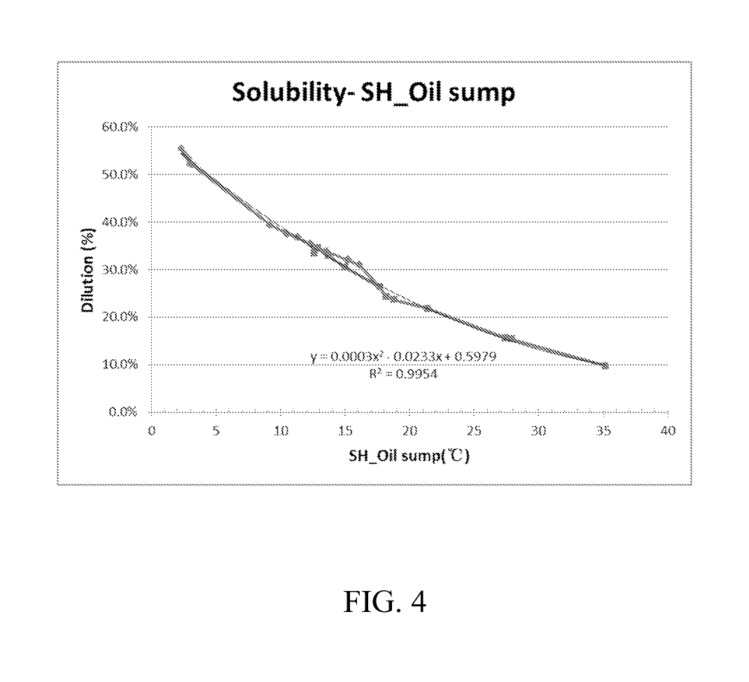

Each point of the oil sump superheat corresponds to one point of the dilution of the lubricating oil, and therefore the relationship curve between the oil sump superheat and the dilution of the lubricating oil may be obtained through fitting, as shown in FIG. 4. By using the relationship curve shown in FIG. 4, an equation is obtained: y=(0.0003x²−0.0233x+0.5979)−a, where y represent the value of dilution, x represents the value of oil sump superheat, and a represent a modification coefficient, and R²=0.9954. It should be noted that the value of “a” may be specifically determined according to a practical operating condition.

It can be understood that the relationship curve and equation shown herein are both for the purpose of description, and those skilled in the art may obtain similar relationship curves and equations according to practical requirements by using the concept shown in the second embodiment of the present invention, rather than being limited to the specific forms in the foregoing.

For implementation convenience, the foregoing equation may be written in relevant software program, and accordingly corresponding dilution of the lubricating oil can be displayed on a control panel of a compressor system or the refrigeration air conditioning system. Certainly, an externally connected display device may also be used as an accessory of the compressor and is installed on the compressor to display the dilution of the lubricating oil.

Under all operating conditions for running the compressor, the solubility of the refrigerant in the lubricating oil (i.e., the dilution of the lubricating oil) may be obtained after the oil sump temperature and the suction/discharge pressure or evaporating/condensing temperature are measured by way of looking up in a table created according to a relationship similar to or same as the relationship curve shown in FIG. 4, and meanwhile, the corresponding oil sump superheat may also be obtained.

### Example 1

Set a low-pressure chamber compressor as an example. It is detected that an oil sump temperature T1 of the compressor is 23.7° C. and midpoint temperature T2 of an evaporator coil is 10° C., and the oil sump superheat of the compressor can be obtained through calculation SH=T1−T2=13.7° C. According to the foregoing curve formula, the value of dilution corresponding to the oil sump superheat SH is obtained by calculation (33.5%−a). The value of dilution of the lubricating oil is the dilution of the lubricating oil in the compressor in a working state.

### Example 2

Set a low-pressure chamber compressor as an example. It is detected that an oil sump temperature T1 of the compressor is 23.7° C. and a suction pressure P2 of the compressor is 9.82 Bar, and a saturation temperature T2 corresponding to P2 can be calculated and is 10° C. according to a thermophysical property equation or diagram of a refrigerant. Oil sump superheat of the compressor may be calculated SH=T1−T2=13.7° C. According to the foregoing curve formula, the value of dilution corresponding to the oil sump superheat SH is obtained through calculation and is (33.5%−a). This value of dilution of lubricating oil is the dilution of the lubricating oil in the compressor in the working state.

### Example 3

Set a high pressure chamber compressor as an example. An oil sump temperature T1 of the compressor and a midpoint temperature of a condenser coil T2 are detected, and oil sump superheat of the compressor can be calculated and is SH=T1−T2. According to the foregoing curve formula, the value of dilution corresponding to the SH is obtained through calculation and is subtracted by a modification coefficient. This value of dilution of lubricating oil is the dilution of the lubricating oil in the compressor in the working state.

### Example 4

Set a high pressure chamber compressor as an example again. The oil sump temperature T1 of the compressor and discharge pressure P2 of the compressor are detected. A saturation temperature T2 corresponding to the discharge pressure P2 can be obtained by calculation according to a thermophysical property equation or diagram of a refrigerant. The oil sump superheat of the compressor is obtained through calculation SH=T1−T2. According to the foregoing curve formula, the value of dilution corresponding to the superheat SH is obtained through calculation and is subtracted by a modification coefficient. This value of dilution of lubricating oil is the dilution of the lubricating oil in the compressor in the working state.

As mentioned above, it can be seen that the method provided in the second embodiment of the present invention is simple, low in cost, and suitable for practical applications in engineering.

### Third Embodiment

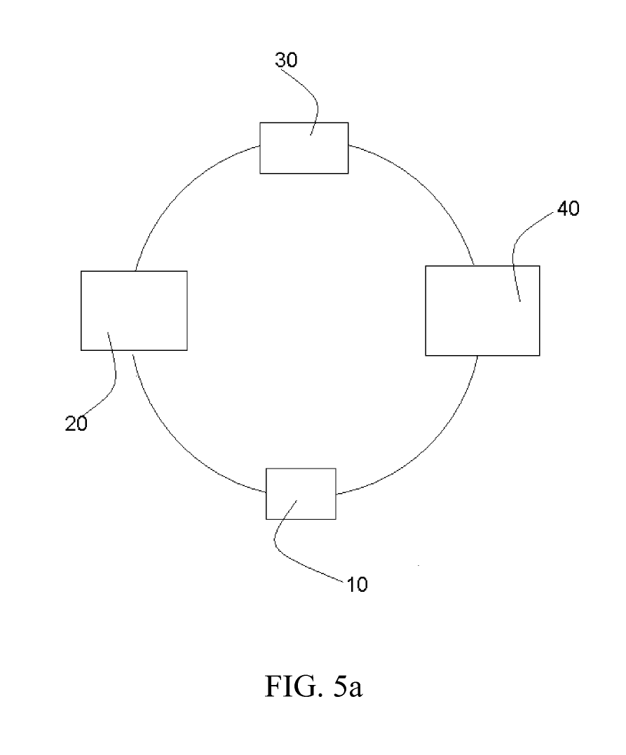

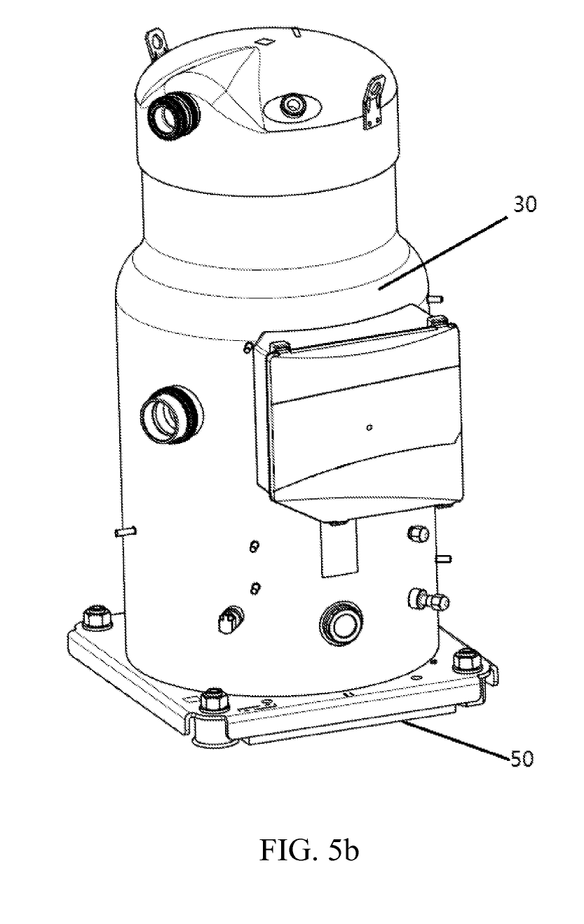

As shown in FIG. 5a, a refrigeration air conditioning system 100 is provided. The refrigeration air conditioning system includes a compressor 30, a condenser 20, a throttling apparatus (e.g., an expansion valve or a capillary tube) 10 and an evaporator 40 sequentially connected through a pipeline. A refrigerant goes through a refrigeration circulation within the refrigeration air conditioning system by evaporation, compression, condensation and throttling.

The compressor sucks in low-pressure and low-temperature refrigerant vapor from the evaporator, and compresses the low-pressure and low-temperature refrigerant vapor into high-temperature and high-pressure refrigerant vapor. Subsequently, the high-temperature and high-pressure refrigerant vapor is condensed into high-temperature and high-pressure refrigerant liquid in the condenser and becomes low-temperature and low-pressure refrigerant liquid after passing through the throttling apparatus. The low-temperature and low-pressure refrigerant liquid is then conveyed into the evaporator and absorbs heat in the evaporator to be evaporated into the low-temperature and low-pressure refrigerant vapor. The low-temperature and low-pressure refrigerant vapor is then delivered to the inlet of the compressor again. Accordingly, a refrigeration circulation is accomplished.

As discussed above, there are some disadvantages to control a crankcase heater according to an ambient temperature, for example, the ambient temperature cannot directly reflect the true viscosity of the lubricating oil, power input of the compressor may be increased and performance of the compressor may be decreased when the crankcase heater is controlled to be turned on or off according to the ambient temperature.

Referring to FIG. 5b, the refrigeration air conditioning system further includes a crankcase heater 50 at the bottom of the compressor 30 (especially, at the bottom of an oil sump of the compressor), and whether the crankcase heater 50 heats the oil sump is controlled according to viscosity of lubricating oil in the oil sump. The crankcase heater 50 is installed outside the oil sump, for example, below the oil sump or around an external sidewall of the oil sump. In this embodiment, whether to turn on or turn off the crankcase heater 50 is decided directly according to the viscosity of the lubricating oil instead of the ambient temperature. In an implementation manner, the viscosity of the lubricating oil may be the viscosity of the lubricating oil in the oil sump of the compressor 30 that is obtained according to the methods in the foregoing first embodiment and/or second embodiment. In an alternative embodiment, the viscosity of the lubricating oil in the oil sump of the compressor 30 can be obtained by the following: an oil sump temperature of the compressor 30 is detected, oil sump pressure of the compressor 30 is detected, and the viscosity of the lubricating oil in the oil sump is obtained according to the oil sump temperature and the oil sump pressure.

In an implementation manner, in the refrigeration air conditioning system, suction/discharge pressure of the compressor 30 and the oil sump temperature or suction temperature of the compressor 30 are measured, and accordingly the viscosity of the lubricating oil may be obtained according to the measured pressure and the measured temperature. For example, at a suction/discharge outlet of the compressor 30 in the refrigeration air conditioning system, suction/discharge pressure is separately measured by using pressure measurement apparatus (not shown). The temperature of the lubricating oil may be detected by a temperature sensor installed inside the oil sump or at a lower end of the outside of the oil sump.

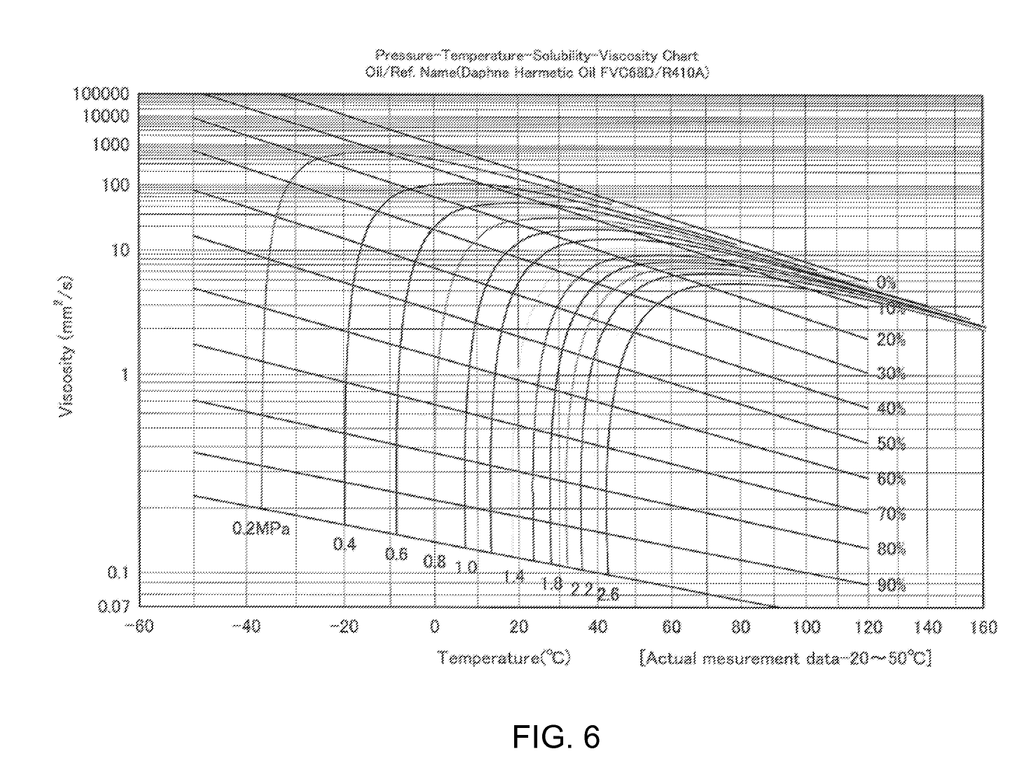

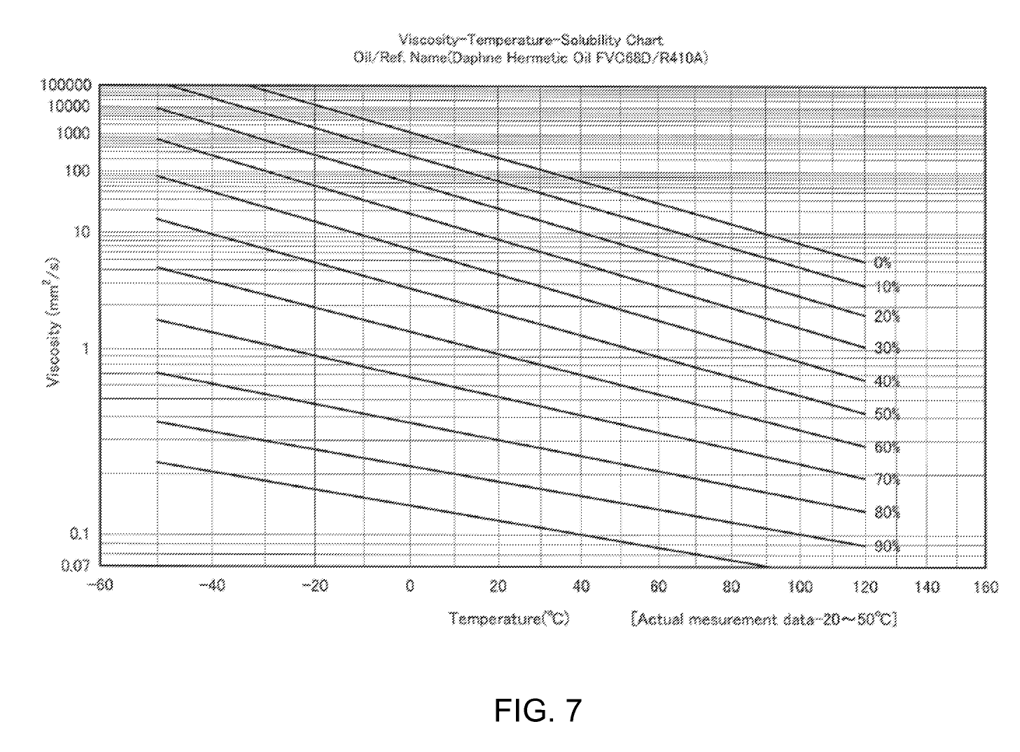

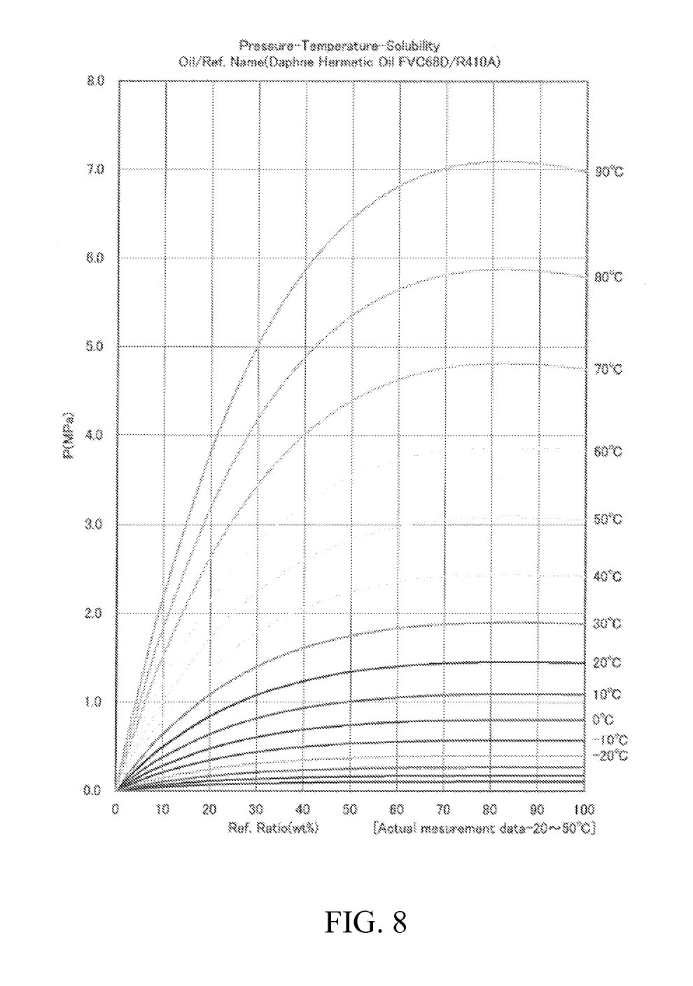

If corresponding oil sump temperature and oil sump pressure of the compressor are obtained under a specific operating condition, the viscosity of the lubricating oil under the specific operating condition is obtained according to a corresponding relationship curve between the dilution and viscosity of the lubricating oil related to the oil sump temperature and pressure (for example, it may be provided by a lubricating oil manufacturer or obtained from a current reference book). In an implementation manner, according to the curve diagram (which may be a curve diagram, which is directly obtained from an oil factory, of temperature, pressure, and solubility of oil) shown in FIG. 8, the solubility of refrigerant may be found according to the oil sump temperature and the pressure, and subsequently, the viscosity of the lubricating oil corresponding to the solubility is obtained according to the curve diagrams shown in FIG. 7 or FIG. 6.

Subsequently, it is determined according to the obtained viscosity of the lubricating oil whether the compressor can run reliably, and it is further decided, by using a controller of the compressor, whether to turn on the crankcase heater 50 to heat the oil sump or alternately whether to stop the compressor according to a preset condition in the controller.

For example, the crankcase heater 50 starts to work when the viscosity of the lubricating oil is less than a preset value.

It can be understood that, in this embodiment, the crankcase heater 50 is directly controlled according to the viscosity of the lubricating oil obtained by using a status (e.g., pressure and temperature) of the lubricating oil. This embodiment can turn on the crankcase heater when the oil sump actually need be heated since this embodiment controls the on/off of the crankcase heater according to the viscosity of the lubricating oil.

As discussed above, the oil sump pressure may be directly obtained by using the measured suction pressure of the compressor. In addition, the oil sump temperature may also be indirectly or approximately obtained from the measured suction/discharge temperature. However, if the compressor is in an off state, the oil sump temperature obtained may not be precise enough, and therefore preferably the oil sump temperature is measured by a temperature sensor disposed inside the oil sump of the compressor or around an external sidewall or at a lower end of the outside of the oil sump in the compressor. In an implementation manner, when the viscosity of the lubricating oil in the oil sump is lower than a preset value, the crankcase heater starts to heat the oil sump to increase the viscosity of the lubricating oil.

Compared with the conventional method for controlling the on/off of the crankcase heater according to an ambient temperature, the method in this embodiment can lower power input of the compressor and improve performance of the compressor.

An embodiment of the present invention may further provide a control module. The control module is operable for controlling a compressor. The control module includes a viscosity obtaining unit, operable for obtaining viscosity of lubricating oil in an oil sump of the compressor, and a control unit, operable for controlling on/off of the compressor 30 according to the viscosity obtained, or controlling on/off of the crankcase heater 50 of the compressor 30 according to the viscosity obtained.

Furthermore, the viscosity obtaining unit includes: a temperature signal receiving unit, operable for receiving an oil sump temperature of the compressor; a pressure signal receiving unit, operable for receiving oil sump pressure of the compressor; and a calculating unit, operable for obtaining, according to the oil sump temperature and the oil sump pressure, the viscosity of the lubricating oil in the oil sump.

It may be understood that, the crankcase heater according to an embodiment of the present invention may be controlled by the control method or the control module herein.

The foregoing provides only some embodiments of the present invention, and those skilled in the art will understand that changes may be made to these embodiments without departing from the principle of the general inventive concept, and the scope of the present invention is defined by the claims and equivalents thereof.

What is claimed is:

1. A method for measuring dilution and/or viscosity of lubricating oil in a compressor, comprising:
detecting a pressure difference between an inlet and an outlet of an oil pump in the compressor by pressure difference detection apparatus, and determining the dilution of the lubricating oil and/or the viscosity of the lubricating oil according to the pressure difference detected; a lower end of the oil pump being located in the lubricating oil in the oil sump, a high-pressure side of the pressure difference detection apparatus being connected to the outlet of the oil pump and a low-pressure side of the pressure difference detection apparatus being connected to bottom of the oil sump.

2. The method according to claim 1, wherein,
the oil pump is a positive displacement oil pump.

3. The method according to claim 1, wherein,
the pressure difference detection apparatus is a pressure difference transmitter, the high-pressure side of the pressure difference transmitter is connected to the outlet of the oil pump through a high-pressure pipe, and a low-pressure side of the pressure differential transmitter is connected to the bottom of the oil sump through a low-pressure pipe.

4. The method according to claim 1, wherein,
the detecting the pressure difference between the inlet and the outlet of the oil pump in the compressor is performed under multiple different operating conditions, and accordingly the determining the dilution and/or the viscosity of the lubricating oil according to the pressure difference is performed respectively under the multiple different operating conditions; wherein the method further comprises:
obtaining a relationship curve between the pressure difference and the dilution of the lubricating oil suitable for the multiple different operating conditions by way of fitting, and/or a relationship curve between the pressure difference and the viscosity of the lubricating oil for the multiple different operating conditions by way of fitting.

5. The method according to claim 4, further comprising:
after detecting the pressure difference between the inlet and the outlet of the oil pump, obtaining the dilution and/or the viscosity of the lubricating oil corresponding to the pressure difference detected according to the relationship curve(s).

6. The method according to claim 4, wherein,
the compressor is a low-pressure chamber compressor or a high-pressure chamber compressor used in a refrigeration air conditioning system.

7. A method for detecting dilution of lubricating oil in a compressor, comprising:
measuring an oil sump temperature of a compressor, an evaporating/condensing temperature of the compressor, and dilution of the lubricating oil under multiple different operating conditions;
calculating oil sump superheat according to an equation that the oil sump superheat=the oil sump temperature−the evaporating/condensing temperature;
obtaining, by way of fitting, a relationship curve between the oil sump superheat and the dilution of the lubricating oil, the relationship curve applicable for the multiple different operating conditions; and
obtaining, according to the relationship curve between the oil sump superheat and the dilution of the lubricating oil, dilution of the lubricating oil corresponding to oil sump superheat obtained under a certain operating condition.

8. The method according to claim 7, wherein,
the condensing temperature of the compressor is a saturation temperature corresponding to discharge pressure of the compressor, and the evaporating temperature of the compressor is a saturation temperature corresponding to suction pressure of the compressor.

9. The method according to claim 8, wherein,
the saturation temperature corresponding to the suction pressure of the compressor or corresponding to the discharge pressure of the compressor is calculated by a thermophysical property equation or diagram of refrigerant in the compressor.

10. The method according to claim 7, wherein,
the evaporating temperature of the compressor is a midpoint temperature of an evaporator coil; and/or,
the condensing temperature of the compressor is midpoint temperature of a condenser coil.

11. The method according to claim 7, wherein,
the relationship curve between the oil sump superheat and the dilution of the lubricating oil is: y=(0.0003x²−0.0233x+0.5979)−a, y represents the dilution, x represents the oil sump superheat, and a represents a modification coefficient.

12. The method according to claim 11, wherein,
the relationship curve between the oil sump superheat and the dilution of the lubricating oil is written in control software of a control panel of the compressor, and the dilution of the lubricating oil is obtained on the control panel according to the oil sump superheat obtained through calculation;
wherein the method further comprises:
displaying by the control panel the dilution of the lubricating oil obtained.

13. The method according to claim 11, wherein,
the relationship curve between the oil sump superheat and the dilution of the lubricating oil is written in control software of a display device, the display device being externally connected to the compressor and serving as an accessory of the compressor;
the method further comprises:
displaying by the display device the dilution of the lubricating oil according to the oil sump superheat obtained through calculation.

14. A control method, operable for controlling a compressor, comprising:
obtaining viscosity of lubricating oil in an oil sump of the compressor; and
controlling the compressor to be turned on or off or controlling a crankcase heater of the compressor to be turned on or off according to the viscosity.

15. The control method according to claim 14, wherein,
the obtaining the viscosity of the lubricating oil in the oil sump of the compressor comprises: measuring an oil sump temperature of the compressor, an evaporating/condensing temperature of the compressor, and dilution of the lubricating oil under multiple different operating conditions; calculating oil sump superheat according to an equation that the oil sump superheat=the oil sump temperature−the evaporating/condensing temperature; obtaining, by way of fitting, a relationship curve between the oil sump superheat and the dilution of the lubricating oil, the relationship curve applicable for the multiple different operating conditions; obtaining, according to the relationship curve between the oil sump superheat and the dilution of the lubricating oil, dilution of the lubricating oil corresponding to oil sump superheat obtained under a certain operating condition;
and obtaining the viscosity of the lubricating oil according to the oil sump superheat and the dilution of the lubricating oil; or,
the obtaining the viscosity of the lubricating oil in the oil sump of the compressor comprises: detecting a pressure difference between an inlet and an outlet of an oil pump in the compressor, and determining the viscosity of the lubricating oil according to the pressure difference detected; a lower end of the oil pump being located in the lubricating oil in the oil sump, a high-pressure side of the pressure difference detection apparatus being connected to the outlet of the oil pump and a low-pressure side of the pressure difference detection apparatus being connected to bottom of the oil sump.

16. The control method according to claim 14, wherein,
the obtaining the viscosity of the lubricating oil in the oil sump of the compressor comprises: detecting an oil sump temperature in the compressor, detecting oil sump pressure in the compressor, and obtaining the viscosity of the lubricating oil in the oil sump according to the oil sump temperature and the oil sump pressure.

17. A control module, operable for controlling a compressor, comprising:
a viscosity obtaining unit, operable for obtaining viscosity of lubricating oil in an oil sump of the compressor; and
a control unit, operable for controlling according to the viscosity of the lubricating oil the compressor to be turned on or off or controlling according to the viscosity of the lubricating oil a crankcase heater of the compressor to be turned on or off.

18. The control module according to claim 17, wherein the viscosity obtaining unit comprises:
a temperature signal receiving unit, operable for receiving an oil sump temperature of the compressor;
a pressure signal receiving unit, operable for receiving oil sump pressure of the compressor; and
a calculation unit, operable for obtaining the viscosity of the lubricating oil in the oil sump according to the oil sump temperature and the oil sump pressure.

19. A refrigeration air conditioning system, wherein the refrigeration air conditioning system comprises a compressor, a condenser, throttling apparatus, and an evaporator which are sequentially connected through a pipeline, and a refrigerant goes through evaporation, compression, condensing, and throttling in the refrigeration air conditioning system in a refrigeration circulation,
wherein the refrigeration air conditioning system further comprises a crankcase heater, and the crankcase heater is controlled to be turned on or off according to viscosity of lubricating oil in an oil sump of the compressor.

20. The refrigeration air conditioning system according to claim 19, wherein,
the refrigeration air conditioning system further comprises a temperature sensor operable for detecting oil sump temperature, and the temperature sensor is installed inside the oil sump or around an external sidewall or at a lower end of the outside of the oil sump.

21. The refrigeration air conditioning system according to claim 20, wherein,
the oil sump temperature is indirectly or approximately obtained from a discharge/suction temperature of the compressor; and/or,
oil sump pressure is directly obtained from suction pressure of the compressor.

22. The refrigeration air conditioning system according to claim 21, wherein,
the viscosity of the lubricating oil in the oil sump is obtained according to the oil sump temperature and the oil sump pressure; the crankcase heater is operable for starting to heat the oil sump to increase the viscosity of the lubricating oil when the viscosity of the lubricating oil in the oil sump is lower than a preset value.
